(19) United States
(12) Patent Application Publication
(10) Pub. No.: US 2020/0046545 A1
(43) Pub. Date: Feb. 13, 2020

(54) WEARABLE SNORE STOPPING DEVICE

(71) Applicant: Akhter Usman, Stamford, CT (US)

(72) Inventor: Akhter Usman, Stamford, CT (US)

(21) Appl. No.: 16/101,458

(22) Filed: Aug. 12, 2018

(51) Int. Cl. A61F 5/56

(52) U.S. Cl. CPC A61F 5/566

(57) ABSTRACT

A device to prevent a user from snoring, the device including a mouth portion, including a tongue depressor disposed on a first surface of the mouth portion to be inserted into a mouth of the user to press a tongue of the user in order to prevent the user from snoring, a loop disposed on a second surface of the mouth portion, and a strap to thread through the loop and to wrap around a head of the user to allow the user to wear the device.

### BACKGROUND

### 1. Field

The present general inventive concept relates generally to a medical device, and particularly, to a wearable snore stopping device.

### 2. Description of the Related Art

Sleep apnea is a condition that plagues many individuals, keeping them from receiving the proper flow of oxygen to the brain and causing snoring. Currently, on the market, there are other products that exist to remedy sleep apnea, but they are often uncomfortable for the user to utilize throughout the night.

Therefore, there is a need for a device that effectively stops a user from snoring, while maintaining the user's comfort.

### SUMMARY

The present general inventive concept provides a device to prevent a user from snoring.

Additional features and utilities of the present general inventive concept will be set forth in part in the description which follows and, in part, will be obvious from the description, or may be learned by practice of the general inventive concept.

The foregoing and/or other features and utilities of the present general inventive concept may be achieved by providing a device to prevent a user from snoring, the device including a mouth portion, including a tongue depressor disposed on a first surface of the mouth portion to be inserted into a mouth of the user to press a tongue of the user in order to prevent the user from snoring, a loop disposed on a second surface of the mouth portion, and a strap to thread through the loop and to wrap around a head of the user to allow the user to wear the device.

The strap may include a first connecting portion disposed on a first side of a first end of the strap, and a second connecting portion disposed on a second side of a second end of the strap, to connect with the first connecting portion when the strap is wrapped around the head of the user.

### BRIEF DESCRIPTION OF THE DRAWINGS

These and/or other features and utilities of the present generally inventive concept will become apparent and more readily appreciated from the following description of the embodiments, taken in conjunction with the accompanying drawings of which:

FIG. 1 illustrates a front angled view of a device to prevent a user from snoring, according to an exemplary embodiment of the present general inventive concept; and

FIG. 2 illustrates a rear angled view of the device to prevent the user from snoring, according to an exemplary embodiment of the present general inventive concept.

### DETAILED DESCRIPTION

Various example embodiments (a.k.a., exemplary embodiments) will now be described more fully with reference to the accompanying drawings in which some example embodiments are illustrated. In the figures, the thicknesses of lines, layers and/or regions may be exaggerated for clarity.

Accordingly, while example embodiments are capable of various modifications and alternative forms, embodiments thereof are shown by way of example in the figures and will herein be described in detail. It should be understood, however, that there is no intent to limit example embodiments to the particular forms disclosed, but on the contrary, example embodiments are to cover all modifications, equivalents, and alternatives falling within the scope of the disclosure. Like numbers refer to like/similar elements throughout the detailed description.

It is understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

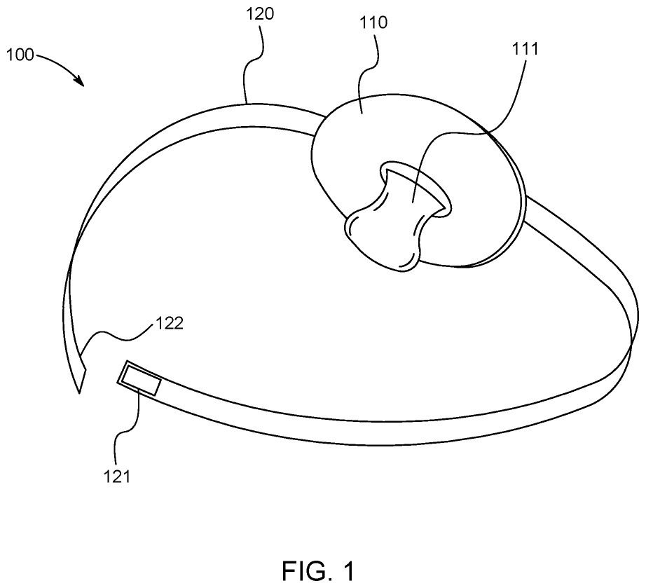

FIG. 1 illustrates a front angled view of a device 100 to prevent a user from snoring, according to an exemplary embodiment of the present general inventive concept.

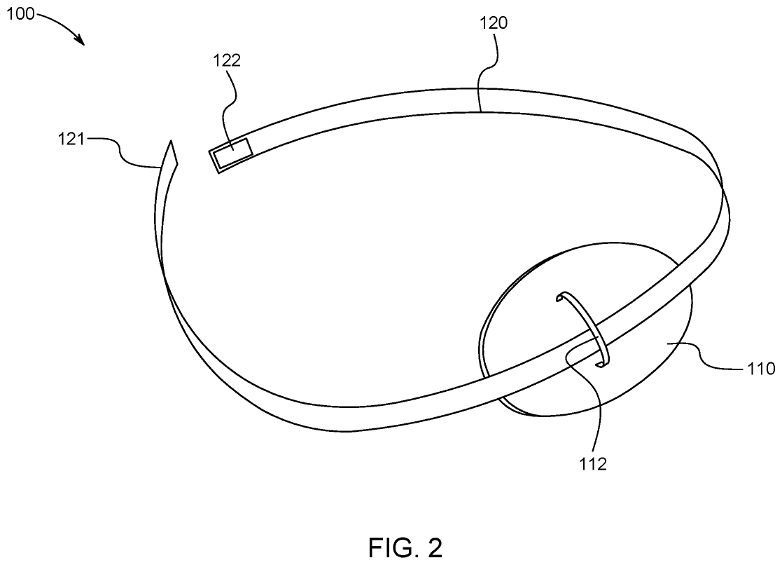

FIG. 2 illustrates a rear angled view of the device 100 to prevent the user from snoring, according to an exemplary embodiment of the present general inventive concept.

The device 100, and components thereof, may have any shape known to one of ordinary skill in the art, may be machine-manufactured (e.g., injection molded), and may be constructed from neoprene, styrofoam, padding, sponge material, plastic, rubber, metal, wood, cloth, etc., but is not limited thereto.

As illustrated in FIGS. 1 and 2, the device 100 may include a mouth portion 110, and a strap 120, but is not limited thereto.

The mouth portion 110 may have a shape similar to an infant's pacifier, and the strap 120 may have a substantially flat and long shape.

The mouth portion 110 may include a tongue depressor 111 and a loop 112.

The tongue depressor 111 may be disposed on a first surface of the mouth portion 110 to extend perpendicularly away therefrom, and may have various shapes and sizes depending on a size of the user's mouth. Also, the tongue depressor 111 may be inserted into the user's mouth to cause a tongue of the user to be held down, thus causing the user to stop snoring.

The loop 112 may be disposed on a second surface of the mouth portion 110, and may be provided to allow the strap 120 to be threaded therethrough, thus allowing the user to wear the device 100. In other words, the loop 112 functions similarly as a belt loop, and allows the strap 120 to be movably connected to the rear surface of the mouth portion 110.

The strap 120 may include a first connecting portion 121 disposed on a first side of a first end of the strap 120, and a second connecting portion 122 disposed on a second side of a second end of the strap 120. Thus, the first connecting portion 121 may connect with the second connecting portion to allow the user to wear the device 100.

The first connecting portion 121 and the second connecting portion 122 may include loops, hooks, adhesives, VELCRO, buttons, apertures, snaps, or any other type of connector known to one of ordinary skill in the art.

Referring to FIGS. 1 and 2, when the user desires to wear the device 100, the user may insert the tongue depressor 111 into the user's mouth, and then may wrap the strap 120 (which is already threaded through the loop 112, and thus, connected to the mouth portion 110) around a head of the user such that the first connecting portion 121 connects to the second connecting portion 122.

Although a few embodiments of the present general inventive concept have been shown and described, it will be appreciated by those skilled in the art that changes may be made in these embodiments without departing from the principles and spirit of the general inventive concept, the scope of which is defined in the appended claims and their equivalents.

1. A device to prevent a user from snoring, the device comprising:
a mouth portion, comprising;
a tongue depressor disposed on a first surface of the mouth portion to be inserted into a mouth of the user to press a tongue of the user in order to prevent the user from snoring,
a loop disposed on a second surface of the mouth portion; and
a strap to thread through the loop and to wrap around a head of the user to allow the user to wear the device.

2. The device of claim 1, wherein the strap comprises:
a first connecting portion disposed on a first side of a first end of the strap; and
a second connecting portion disposed on a second side of a second end of the strap, to connect with the first connecting portion when the strap is wrapped around the head of the user.
